(19) United States
(12) Patent Application Publication
(10) Pub. No.: US 2004/0214210 A1
(43) Pub. Date: Oct. 28, 2004

(54) Method of cRNA synthesis

(75) Inventors: Yanxiang Cao, Mountain View, CA (US); Carsten Rosenow, Redwood City, CA (US); Thomas R. Gingeras, Encinitas, CA (US); David J. Lockhart, Del Mar, CA (US)

Correspondence Address: BANNER & WITCOFF LTD.,, ATTORNEYS FOR AFFYMETRIX, 1001 G STREET , N.W., ELEVENTH FLOOR, WASHINGTON, DC, 20001-4597, US

(73) Assignee: Affymetrix, Inc., Santa Clara, CA

(21) Appl. No.: 10/763,414

(22) Filed: Jan. 26, 2004

Related U.S. Application Data
(63) Continuation of application No. 09/738,892, filed on Dec. 18, 2000.
(60) Provisional application No. 60/172,340, filed on Dec. 16, 1999.

(51) Int. Cl. C12Q 1/68; C12P 19/34

(52) U.S. Cl. 435/6; 435/91.2

(57) ABSTRACT

Copy or antisense RNA is synthesized in a way that preserves the relative amounts of particular RNA molecules in a population. RNA is reverse transcribed to form first strand cDNA. Second strand cDNA is made by enzyme-catalyzed DNA synthesis using exogenous primers, preferably random primers. Double-stranded cDNA formed from the first and second strands is then used as a template for in vitro transcription to form copy or antisense RNA. The transcription is driven by a promoter sequence which was introduced into the cDNA by the primers used for reverse transcription.

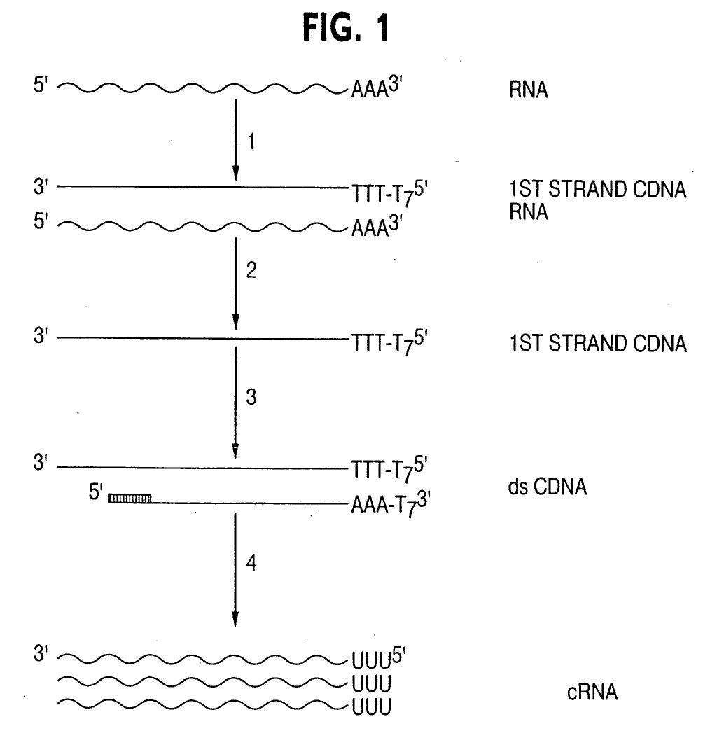

This application claims the benefit of provisional application 60/172,340 filed Dec. 16, 1999, which is expressly incorporated herein.

### TECHNICAL FIELD OF THE INVENTION

The invention relates to the field of expression monitoring. More particularly it relates to the field of determining expression of particular genes as reflected by their respective mRNA species.

### BACKGROUND OF THE INVENTION

Many biological functions are carried out by regulating the expression levels of various genes, either through changes in copy number of a gene, through changes in levels of transcription of particular genes, or through changes in synthesis of particular proteins. Processes such as cell cycle, cell differentiation, and infection are characterized by variations in transcription levels of particular genes and sets of genes.

Massive parallel gene expression monitoring methods have been developed to monitor the expression of a large number of genes using nucleic acid array technology. See, e.g., U.S. Pat. No. 5,871,928; de Saizieu, et al., 1998, Bacterial Transcript Imagining by Hybridization of total RNA to Oligonucleotide Arrays, Nature Biotechnology, 16:45-48; Wodicka et al., 1997, Genome-wide Expression Monitoring in Saccharomyces cerevisiae, Nature Biotechnology 15:1359-1367; Lockhart et al., 1996, Expression Monitoring by Hybridization to High Density Oligonucleotide Arrays. Nature Biotechnology 14:1675-1680; Lander, 1999, Array of Hope, Nature-Genetics, 21 (suppl.), at 3.

Various techniques for making arrays are known. See, U.S. Pat. Nos.: 5,143,854, 5,242,979, 5,252,743, 5,324,663, 5,384,261, 5,405,783, 5,412,087, 5,424,186, 5,445,934, 5,451,683, 5,482,867, 5,489,678, 5,491,074, 5,510,270, 5,527,681, 5,550,215, 5,571,639, 5,593,839, 5,599,695, 5,624,711, 5,631,734, 5,677,195, 5,744,101, 5,744,305, 5,753,788, 5,770,456, 5,831,070 and 5,856,011, each of which is expressly incorporated herein.

Methods for preparing cDNA from mRNAs of a target sample are described in, for example, Lockhart et al., 1996, Expression Monitoring by Hybridization to High Density Oligonucleotide Arrays. Nature Biotechnology 14:1675-1680, U.S. Pat. Nos. 5,716,785 and 5,891,636, which are incorporated by reference for all purposes.

One method for monitoring gene expression employs reverse transcription of templates consisting of mRNAs from target samples to generate a cDNA population. The cDNA molecules then serve as templates for transcription to form cRNA which can be used to hybridize to an array of oligonucleotides. There is a need in the art for additional methods of preparing transcription indicators of cells.

### SUMMARY OF THE INVENTION

It is an object of the invention to provide a method for preparing a population of cRNA.

It is another object of the invention to provide a method for determining expression of a plurality of mRNA species in a biological sample.

These and other objects of the invention are provided by one or more of the following embodiments. In one embodiment of the invention a method is provided for preparing a population of cRNA which represents expression of cells in a sample. A population of mRNA derived from cells in a sample is reverse transcribed to form a population of first strand cDNA hybridized to said mRNA. The reverse transcription employs a primer having a first portion which is complementary to a plurality of mRNA molecules in said population, and a second portion which is a promoter sequence. The first portion is 3′ to the second portion. The promoter sequence is in an antisense orientation with respect to the mRNA. Hybrids of the first strand cDNA and the MRNA are then denatured. A population of random oligomers is hybridized to the population of first strand cDNA. Second strand cDNA complementary to the population of first strand cDNA is synthesized by extending the random oligomers to form a population of double stranded cDNA each having a first and a second strand of cDNA. The first strand of cDNA comprises the promoter sequence at its 5′ end. Double stranded cDNA of said population is transcribed using an RNA polymerase to form a population of cRNA which is antisense with respect to mRNA in said population of mRNA. The population of cRNA represents expression of cells in the sample.

According to another embodiment of the invention another method is provided for preparing a population of cRNA which represents expression of cells in a sample. A population of mRNA derived from cells in a sample is reverse transcribed to form a population of first strand cDNA hybridized to said MRNA. The reverse transcribing employs a primer having a first portion which is polydeoxythymidylate and a second portion which is a phage promoter sequence. The first portion is 3′ to the second portion and the promoter sequence is in an antisense orientation with respect to the mRNA. Hybrids of the first strand cDNA and said MRNA are heat denatured. A population of random oligomer primers is hybridized to the first strand cDNA population. Second strand cDNA is synthesized by extending the population of random oligomer primers to form a population of double stranded cDNA each having a first and a second strand of cDNA. The first strand of cDNA comprises the promoter sequence at its 5′ end. Double stranded cDNA of said population is transcribed using an RNA polymerase to form a population of cRNA which is antisense with respect to MRNA in the population of mRNA. The population of cRNA represents expression of cells in the sample.

According to still another embodiment of the invention a method for determining expression of a plurality of mRNA species in a biological sample is provided. A population of mRNA derived from cells in a sample is reverse transcribed to form a population of first strand cDNA hybridized to said MRNA. The reverse transcribing employs a primer having a first portion which is polydeoxythymidylate, and a second portion which is a phage promoter sequence; the first portion is 3′ to the second portion and the promoter sequence is in an antisense orientation with respect to the MRNA. Hybrids of the first strand cDNA and the mRNA are heat denatured. A population of random hexamer primers is hybridized to the first strand cDNA population. Second strand cDNA is synthesized by extending the population of random oligomer primers, to form a population of double stranded cDNA each having a first and a second strand of cDNA. The first strand of cDNA comprises the promoter sequence at its 5′ end. Double stranded cDNA of the population is transcribed using an RNA polymerase to form a population of cRNA which is antisense with respect to mRNA in the population of mRNA. The population of cRNA represents expression of cells in the sample. A plurality of cRNA species in the population is quantitated by hybridization to an array of oligonucleotide probes.

The invention thus provides the art with suitable means for preparing cRNA for measuring transcript levels in cell populations.

### BRIEF DESCRIPTION OF THE DRAWINGS

The figure schematically shows a method according to the present invention.

### DETAILED DESCRIPTION OF THE INVENTION

It is a discovery of the present inventors that exogenous random oligomer primers can be used to synthesize second strand cDNA synthesis in an overall method of forming cRNA. The ability to utilize “off the shelf” primers avoids the need in prior art methods of using additional enzymatic reactions such as RNase H or S1 nuclease digestions. Avoiding additional enzymatic reactions is beneficial from time, efficiency, and cost perspectives.

Random oligomer primers for use in the present invention can be custom made, “off the shelf” or “home” made. The primers can be from about 6 to about 15 nucleotides in length. The amount of primer used will affect efficiency and the length of synthesized products. The range of weight ratios of hexamer to initial RNA input should be between about 1:100 and 10:1, preferably about 1:10. Higher ratios tend to yield shorter products. Enzymes which can be used to synthesize second strand cDNA are any known in the art for such purpose. E. coli DNA polymerase I can be used, as well as Klenow fragment. These can optionally be used with DNA ligase which will promote longer products.

Reverse transcription is performed in the method of the invention according to standard techniques known in the art. The reaction is typically catalyzed by an enzyme from a retrovirus, which is competent to synthesize DNA from an RNA template. According to the present method, the primer used for reverse transcription has two parts: one part for annealing to the RNA molecules in the cell sample through complementarity and a second part comprising a strong promoter sequence. Typically the strong promoter is from a bacteriophage, such as SP6, T7 or T3. Promoters which drive robust in vitro transcription are desirable. Because most populations of MRNA from biological samples do not share any sequence homology other than a poly(da) tract at the 3′ end, the first part of the primer typically comprises a poly(dT) sequence which is generally complementary to most mRNA species. The length of the tract is typically from about 5 to 20 nucleotides, more preferably about 10 to 15 nucleotides. Alternatively, if a subpopulation of RNA is desired, a primer which is complementary to a common sequence feature in the subpopulation can be used. Yet another type of priming employs random oligomers. Such oligomers should yield a full and representative set of cDNA. The orientation of the promoter sequence is important. It is typically at the 5′ end of the primer, so that the 3′ end can successfully anneal and drive reverse transcription. Moreover, the promoter sequence is oriented in such a fashion that it is “opposite” the 3′ end of the MRNA. Thus upon second strand synthesis, the double stranded promoter will be at the 3′ end of the gene, in an orientation favorable for producing reverse strand (negative strand, or antisense) RNA. This orientation is termed “antisense” orientation. Hybrids of first strand cDNA and MRNA can be denatured according to any method known in the art. These include the use of heat and the use of alkali. Heat treatment is the preferred method. Denaturation is desirable until less than 50% of the hybrids remain annealed. More denaturation is desirable, such as until less than 75%, 85% or 95% of the hybrids remain annealed as hybrids.

Transcription of the double stranded cDNA molecules is a linear process which creates large amounts of product from small input amount, without greatly distorting the relative amounts of input. Thus the transcription process while being efficient is “linear” rather than “exponential.” Labeled ribonucleotides can be used during transcription of the double stranded cDNA. These can be radioactively labeled, with such isotopes as ³²P, ³H, and ³²S. Fluorescently labeled ribonucleotides can also be used. Biotin labeled nucleotides can also be used. Subsequent to incorporation, labeled avidin can be bound to biotin-labeled polynucleotides. The labeled avidin can contain any desirable and convenient detectable label.

Quantitation of particular RNA molecules within the population of copy RNA can be done according to any means known in the art. These include but are not limited to Northern blotting and hybridization to nucleic acid arrays. Typically, some sort of hybridization step must be involved to provide the specificity required to measure transcripts individually. Alternatively, the cRNA can be reverse transcribed into cDNA and a specific cDNA species can be amplified to obtain specificity. Copy RNA can be used for any use known in the art, not merely quantitation. It can be used for cloning, and/or expression, or as a probe. Such uses can be applied to determining a diagnosis or prognosis, to determining an etiological basis for disease, for determining a cell type or species source, for identifying infectious organisms in foods, hospitals, ventilation systems, and for testing drugs for their main or side effects. Other applications will be readily apparent to those of skill in the art.

The following example is just one among many possible ways to practice the present invention: The invention is not limited in scope to the example.

### EXAMPLE

Random Primer—Antisense RNA Amplification Protocol (biotin-labeling)

Step 1:First Strand DNA Synthesis

Use Gibco-BRL's Superscript Choice System

| | |
|---|---|
| Total RNA (5 μg) | up to |
| | 10 μl |
| T7-(dT)24 oligo (100 pmol) | 1 μl |
| 5× First strand cDNA buffer | 4 μl |
| 0.1 M DTT | 2 μl |
| 10 mM dNTP mix | 1 μl |
| Superscript II RT (200 U/μl) | 2 μl |

Incubate at 42° C. for 1 hr.

Step 2:Second Strand cDNA Synthesis

Use Gibco-BRL's Superscript choice System

Boil the tube at 99° C. for 5 min, chill on ice immediately, Quickly spin down;

Add:

| | |
|---|---|
| DEPC H₂O | 43 μl |
| 10× T4 DNA Poly. buffer | 10 μl |
| 10 mM dNTP mix | 3 μl |
| Random hexamer (50 ng/μl) | 20 μl |
| Klenow (2 U/μl) | 2 μl |
| T4 DNA polymerase (5 U/μl) | 2 μl |

Mix/spin down/incubate at 37° C. for 2 hr.

Step 3: Clean up of ds DNA

Add 1 μl glycogen, 0.6 vol. Of 5.0M NH4OAC and 2.5 vol. Of cold absolute ethanol and vortex.

Immediately centrifuge at full speed for 20 min at room temperature.

Wash pellet with 0.5 ml of 80% ethanol. Centrifuge at maximum speed at room temperature for 5 min.

Step 4: Antisense RNA Amplification

Use BioArray High yield RNA Transcript Labeling Kit from Enzo (P/N 900182)

Incubate 4-6 hours at 37° C.

Step 5: Clean-up aRNA

Use Rneasay spin columns from QIAGEN (P/N 74103). Follow Rneasy Protocol for RNA Clean-up from he QIAGEN handbook.

The above description is illustrative and not restrictive. Many variations of the invention will be apparent to those of skill in the art upon reviewing the above description. The scope of the invention should therefore be determined by the claims, along with the full scope of equivalents to which such claims are entitled.

1. A method for analyzing a population of RNA comprising the steps of:
producing a population of cDNA from a population of RNA, wherein said method employs a primer having a first portion which is complementary to a plurality of RNA molecules in said population, and a second portion comprising a promoter sequence, wherein the first portion is 3′ to said second portion;
synthesizing second strand cDNA complementary to said population of first strand cDNA by extending random oligomers, to form a population of double stranded cDNA;
creating a population of cRNA from said double stranded cDNA;
hybridizing the population of cRNA to an array; and
analyzing a resulting hybridization pattern.

2. The method of claim 1, wherein said population of RNA comprises poly(A+) RNA.

3. The method of claim 2, wherein said poly(A+) RNA comprises MRNA.

4. The method of claim 1, wherein said population of RNA comprises total RNA.

5. The method of claim 1, wherein said promoter sequence comprises a phage T3 promoter.

6. The method of claim 1, wherein said promoter sequence comprises a phage SP6 promoter.

7. The method of claim 1, wherein said promoter sequence comprises a phage T7 promoter.

8. The method of claim 1, wherein said random oligomers are of a uniform length.

9. The method of claim 1, wherein said random oligomers comprise hexamers.

10. The method of claim 1, wherein said random oligomers are between 6 and 15 nucleotides in length.

11. The method of claim 1, wherein said second strand cDNA is synthesized using the Klenow fragment of DNA polymerase I.

12. The method of claim 1, wherein said second strand cDNA is synthesized using T4 DNA polymerase.

13. The method of claim 1, wherein said second strand cDNA is synthesized using E. coli DNA polymerase I alone or in conjunction with a DNA ligase.

14. The method of claim 1, wherein said first portion of said primer comprises a poly deoxythymidylate (poly dT) sequence.

15. A method for analyzing a population of RNA comprising the steps of:
producing a population of cDNA from a population of RNA, wherein said method employs a primer having a first portion comprising oligo dT, and a second portion comprising a phage promoter sequence, wherein the first portion is 3′ to said second portion;
synthesizing second strand CDNA complementary to said population of first strand cDNA by extending random oligomers, to form a population of double stranded cDNA;
creating a population of cRNA from said double stranded cDNA;
hybridizing the population of cRNA to an array; and
analyzing a resulting hybridization pattern.

16. The method of claim 1, wherein said population of cRNA is synthesized using an RNA polymerase.

17. The method of claim 15, wherein said population of cRNA is synthesized using an RNA polymerase.

18. The method of claim 2, wherein said RNA is isolated from an eukaryotic cell or tissue.

19. The method of claim 18, wherein said eukaryotic cell or tissue is mammalian.

20. The method of claim 19, wherein said mammalian cell or tissue is human.
